(19) United States
(12) Patent Application Publication
(10) Pub. No.: US 2011/0223221 A1
(43) Pub. Date: Sep. 15, 2011

(54) STABILIZATION PROCESSES OF CATION RADICALS OF PHENOTHIAZINIC COMPOUNDS, COSMECEUTICAL FORMULATIONS AND METHODS FOR SKIN DISEASES AND DISTURBANCES PREVENTION

(76) Inventors: Iseli Lourenco Nantes, Mogi das Cruzes -SP (BR); Tiago Rodrigues, Mogi das Cruzes -SP (BR); Carolina Gregorutti Dos Santos, Mogi das Cruzes-SP (BR)

(21) Appl. No.: 12/992,291

(22) PCT Filed: May 12, 2009
(86) PCT No.: PCT/BR2009/000126

§ 371 Date: May 18, 2011

(30) Foreign Application Priority Data
May 12, 2008 (BR) PI0801368-3

(51) Int. Cl. A61K 8/49; C07D 417/06; A61K 31/5415; A61K 8/02; A61K 9/127; A61K 9/14; A61P 17/00; A61P 17/02; A61P 35/00; A61Q 1/00; A61Q 17/04; A61P 19/08

(52) U.S. Cl. 424/401; 544/44; 544/42; 514/225.5; 514/225.8; 424/59; 424/450; 424/400

(57) ABSTRACT

The present invention relates to processes of stabilization of cation radicals from one or more phenothiazinic compounds or derivatived compounds thereof. Another embodiment of the present invention relates to cosmeceutical formulations comprising one or more phenothiazinic compounds or derivatived compounds thereof, in combination with cosmeceutically acceptable excipients. The invention also relates to uses of one or more phenothiazinic compounds or derivatived compounds thereof, i the preparation of cosmeceutical formulations for the prevention of skin diseases and disturbances. Methods for prevention of skin diseases and disturbances are also disclosed by the present invention. Particularly, the embodiments of the present invention employ the cited one or more phenothiazinic compounds o derivatived compounds thereof in the form of their stabilized cation radicals.

### FIELD OF THE TECHNIQUE

The present invention relates to photoprotective cosmeceutical formulations comprising phenothiazines as active principle, associated or not to cosmeceutical assistants.

### BACKGROUND OF THE INVENTION

### Cosmetic Industry and Cosmeceutics

According to ANVISA and the Brazilian Ministry of Health, the cosmetics comprise the personal hygiene products, the cosmetic products, the perfumes and the substances or compositions containing natural and synthetic substances, and mixtures thereof, for external use in diverse parts of the human body, of the skin, of the capillary system, of the nails, of the lips, of the external genital organs, of the teeth and of the mucous membranes of the oral cavity, with the exclusive or the main purpose of cleaning, aromatizing, changing the appearance and/or correcting body odors and/or protecting them and keeping them in good conditions (RDC ANVISA no. 211/05). This is also the definition adopted by the Mercosul and the European Union (ABIHPEC—Brazilian Association of the Personal Hygiene, Fragrance and Cosmetic Industry: http://www.abihpec.org.br/areatecnica_regbrasil2.php.

The Brazilian cosmetic market closed the year of 2006 with liquid earnings of US$8.1 billions (about R$17.5 billions), according to ABIHPEC. Comparing to the world market of Personal Hygiene, Fragrances and Cosmetics, according to the data for 2006 of Euromonitor, Brazil is the third of the ranking. It is the second market in baby products, deodorants and fragrances; third in bath products, men products, oral hygiene and hair products; fourth in coloring cosmetics; fifth in sunscreen; eight in skin products, and ninth in waxing products (ABIHPEC—Brazilian Association of the Personal Hygiene, Fragrance and Cosmetic Industry: http://www.abihpec.org.br/conteudo/material/apresentacaosetor_{—}2006_{—}2007. pdf).

The search for cosmetics with therapeutic purposes, from the use of chemical active principles, made common the adoption of a new denomination: the cosmetics, understood as a fusion between the pharmaceutical and cosmetic industries (Pachione, R. “Cosméticos” Journal Química e Derivados no. 445, January 2006).

The increasing tendency for the cosmetic products, besides the creation of new segments of markets, intensifies the importance of technological factors in the production and competition standards in the area and the necessity of rigidity of the legislation (Marinho, V. M. C. “Como as empresas brasileiras de cosméticos estão utilizando o conhecimento tradicional e as plantas medicinais” XXVI RESEM—Annual Reunion about Micromolecular Evolution, Systematics and Ecology, Chemistry Institute of Federal University of the State of Rio de Janeiro (UFF), P37, December 2004).

This area is very expressive in The United States of America, in Europe and in Japan and has been increasing in the Brazilian market. The cosmeceutics represents more than US$50 billions of the global business of cosmetic (Pachione, R. “Cosméticos” Journal Química e Derivados no. 445 January 2006).

Solar Radiation Action

The Sun is essential to life on Earth and its effects on men depend on the individual characteristics of the exposed skin, of the intensity of the radiation and of the frequency and time of skin exposure to the radiation. Such factors depend on the geographic localization, the season of the year, the period of the day and the climate conditions. The said effects bring benefits to the human being, as the well-being physical and mental sensation, the melanin production stimulus, leading to skin tanning, the treatment for jaundice (yellowish color of skin and eyes caused by the excess of bilirubin in the blood) and others. On the other hand, the solar radiation can cause, still, damage to the organism if the appropriate caution regarding the amount of solar radiation exposition is not taken (De Paola, M. V. R. V., Ribeiro, M. E. “Interação entre filtros solares” Cosmetics & Toiletries, September-October 1998 apud Flor, J. et al. “Protetores Solares” Química Nova, v. 30, 2007).

The solar spectrum that reaches the Earth surface is formed predominantly by ultraviolet radiations (100-400 nm), visible (400-800 nm) and infrared (above 800 nm). The human organism fells the presence of these radiations of the solar spectrum in different ways. The infrared radiation (IR) is perceived in the form of heat, the visible radiation (Vis) is perceived through the different colors detected by optical system and the ultraviolet radiation (UV) is perceived through photochemical reactions. Such reactions can stimulate the melanin production, which manifestation is visible in the form of skin tanning, or can lead to the production of simple inflammations up to severe burns. There is still the possibility of occurrence of genetic mutations and abnormal behavior of cells, which frequency has been increasing in the recent years (Osterwalder, U. et al. “Novo Protetor UVA” Cosmetics & Toiletries, July-August 2000 apud Flor, J. et al. “Protetores Solares” Química Nova, v. 30, 2007).

The energy of solar radiation increases with the reduction of the wavelength. This way, the UV radiation is of the shorter wavelength and, consequently, the most energetic, being the most prone to induce photochemical reactions. Other important consideration is regarding the capacity of this radiation to penetrate the skin structure. The UV radiation of lower energy penetrates the deepest in the skin and, when it reaches the dermis, is responsible for the photoaging (Thomas, M. “Ultraviolet and Visible Spectroscopy” 2^{nd} ed., Wiley, 2000 apud Flor, J. et al. “Protetores Solares” Química Nova, v. 30, 2007).

The UV radiation range (100 to 400 nm) can be divided in three parts: UVA, UVB and UVC (Ruvolo Júnior, E. C., Cosméticos On Line, 19, 1997 apud Flor, J. et al. “Protetores Solares” Química Nova, v. 30, 2007).

The UVA radiation (320 to 400 nm)—Frequently, the UVA radiation does not cause erythema. Depending on the skin and the intensity of radiation received, the caused erythema is minimal. When compared to UVB radiation, its capacity to induce erythema in human skin is approximately a thousand times lower. However, it penetrates more deeply in the dermis and induce skin pigmentation, promoting tanning by means of darkening of melanin, through the photooxidation of leucomelanin localized in the cells of the external layers of epidermis (Osterwalder, U. et al. “Novo Protetor UVA”Cosmetics & Toiletries, July-August 2000 apud Flor, J. et al. “Protetores Solares” Química Nova, v. 30, 2007). The UVA radiation is more abundant than the UVB radiation in the Earth surface (UVA 95%, UVB 5%). Histologically, it causes damages to the peripherical vascular system and induces skin cancer, depending on the type of skin and time, frequency and intensity of the exposure (Ruvolo Júior, E. C., Cosméticos On Line, 19, 1997 apud Flor, J. et al. “Protetores Solares” Química Nova, v. 30, 2007; Steiner, D., Cosmetics & Toiletries, 1995 apud Flor, J. et al. “Protetores Solares” Química Nova, v. 30, 2007). The UVA radiation also can act in an indirect way, generating free radicals (Osterwalder, U. et al. “Novo Protetor UVA” Cosmetics & Toiletries, July-ago 2000 apud Flor, J. et al. “Protetores Solares” Química Nova, v. 30, 2007). With the passing years, it causes alterations of collagen and elastic fibers, favoring precocious aging (Billhimer, W. L. “Avaliação de filtros solares em seres humanos: proteção contra a queimadura solar” Cosmetics & Toiletries, 1989 apud Ribeiro, R. P. et al. “Avaliação do Fator de Proteção Solar (FPS) in vitro de produtos comerciais e em fase de desenvolvimento” Journal Infarma, v. 16, 2004).

UVB radiation (280 to 320 nm)—The UVB radiation reaches all Earth surface after passing through atmosphere. It has high energy and, in great frequency, causes sunburns. It also induce skin tanning, being responsible for the transformation of the epidermal ergosterol into vitamin D, and causes the precocious aging of the cells (Ruvolo Júnior, E. C., Cosméticos On Line, 19, 1997 apud Flor, J. et al. “Protetores Solares” Química Nova, v. 30, 2007; Steiner, D., Cosmetics & Toiletries, 1995 apud Flor, J. et al. “Protetores Solares” Química Nova, v. 30, 2007). The frequent and intense exposition to UVB radiation can cause lesion in the DNA, besides suppressing the immunologic response of the skin. This way, besides increasing the risk of fatal mutations, manifested in the form of skin cancer, its activity reduce the chance of a malignant cell to be recognized and destroyed by the organism (Streilein, J. W. et al. “Immune surveillance and sunlight-induced skin cancer” Immunology Today, 15, 1994 apud Flor, J. et al. “Protetores Solares” Química Nova, v. 30, 2007).

UVC radiation (100 to 280 nm)—The UVC radiation is the carrier of high energies, a characteristics that makes it extremely harmful do live beings (Steiner, D., Cosmetics & Toiletries, 1995 apud Flor, J. et al. “Protetores Solares” Química Nova, v. 30, 2007; Streilein, J. W. et al. “Immune surveillance and sunlight-induced skin cancer” Immunology Today, 15, 1994 apud Flor, J. et al. “Protetores Solares” Química Nova, v. 30, 2007). Due to the absorption by the oxygen and ozone in the stratosphere, no UVC radiation and a small fraction of UVB reaches the Earth surface. Due to environmental factors, the reduction of the ozone layer has been leading to an increase of the UVB radiation in the Earth surface, causing higher incidence of burns and, consequently, skin cancer (Roy, C. R. et al. “The solar UV radiation environment: measurement techniques and results” J. Photochem. Photobiol., 31, 1995 apud Flor, J. et al. “Protetores Solares” Química Nova, v. 30, 2007). Australia has been having great problems with the levels of to ultraviolet radiation due to its localization and to the large-scale destruction of the ozone layer in Antarctica (Roy, C. R. et al. “The solar UV radiation environment: measurement techniques and results” J. Photochem. Photobiol., 31, 1995 apud Flor, J. et al. “Protetores Solares” Química Nova, v. 30, 2007), causing an higher incidence of skin cancer (Giles, G, “Report of the National health and medical research council” Australian Government Printing Service, 1989 apud Flor, J. et al. “Protetores Solares” Química Nova, v. 30, 2007; Marks, R. “Report of the National health and medical research council” Australian Government Printing Service, 1989 apud Flor, J. et al. “Protetores Solares” Química Nova, v. 30, 2007).

Sunscreens

The harms to the health, related to UV radiation, can be minimized by the employment of sunscreens (Taylor, C. R. et al. “Photoaging/photodamage and photoprotection” J. Am. Acad. Dermatol., 22, 1990 apud Flor, J. et al. “Protetores Solares” Química Nova, v. 30, 2007), which are available in the market for more than 60 years.

The sunscreen emerged when it was observed that there were substances capable of preventing skin burns (erythema) by the solar rays. In the beginning of the last century, it was observed that acidic quinine sulfate and, later, the Antilux® (2-naphthol-6,8-sodium disulfonate) prevented such effects (Urbach, F. “The historical aspects of sunscreens” J. Photoch. Photobio. B., v. 64, 2001 apud Flor, J. et al. “Protetores Solares” Química Nova, v. 30, 2007). In the end of the twentieth century, many substances arose with efficacy to prevent solar erythema and its use has became more popular, after the World War II, with the employment of the p-amine benzoic acid (PABA) (Shaath, N. A. “Evolution of moderns sunscreen chemicals” Sunscreens, Development, Evaluation and Regulatory Aspects, 1997 apud Flor, J. et al. “Protetores Solares” Química Nova, v. 30, 2007).

Initially, they were developed to protect the skin against sunburns, which is, preferentially against UVB radiation, allowing tanning by means of UVA. With the increasing knowledge regarding UVA radiation, it became evident that the skin would need to be protected against all UVA/UVB range, in order to reduce the skin cancer risk caused by sun exposition (Ziegler, A. et al., Nature, 372, 1994; Ananthaswsmy, H. N. et al., Nat. Med., 3, 1997 apud Flor, J. et al. “Protetores Solares” Química Nova, v. 30, 2007).

Consequently, a new concept was born: an efficient sunscreen must prevent not only a possible sunburn, but must also reduce the accumulation of lesions induced by UV radiation, which could increase the risk of fatal alterations (Schueller, R. et al. “Introdução aos Produtos Fotoprotetores” Cosmetics & Toiletries, 2000 apud Flor, J. et al. “Protetores Solares” Química Nova, v. 30, 2007). To protect the skin from the manifestations produced by UV radiation means to convert its energy in other forms of energy and to guarantee that this other form is not harmful to the skin. The UV filters to employed in formulations of sunscreens need to be chemically and photochemically inert (Osterwalder, U. et al. “Novo Protetor UVA” Cosmetics & Toiletries, July-ago 2000 apud Flor, J. et al. “Protetores Solares” Química Nova, v. 30, 2007).

The need for the use of sunscreens, also named photoprotectors, is a certain fact and, following this trend, the market offers its response. It is estimated that, in 1992, the Brazilian market of sunscreens has commercialized 650 tons of the products. Ten years later, in 2002, this same market reached the production of approximately 4,200 tons. Such numbers not only reveal the increasing importance of this segment, but also suggest the enormous potential of increase for the next years. The fact that the global market dealt, in 2002, with US$3.45 billions in transactions and that, of this total, Latin America had contributed with only US$247.6 millions reinforces this potential (“Dossiê especial sobre o sol” Cosméticos e Perfumes, 27, 2003 apud Flor, S. et al. “Protetores Solares” Química Nova, v. 30, 2007).

Besides the marketable aspect, the great focus for this sector is based for sure in real need of photoprotection. An study performed recently by the American organization EWG (Environmental Working Group), with 785 sunscreens available in the North American market demonstrated that 84% of the tested sunscreens with solar protection factor 15 or above offer inadequate protection to UV radiation (EWG databank: www.ewg.org/sunscreen/ apud Flor, J. et al. “Protetores Solares” Química Nova, v. 30, 2007). In this meaning and with the purpose of offering preparations with higher efficacy—products with better protection efficiency, higher chemical stability and more accessible to the general public—the segment has been exigent to the formulation makers for high technical skills and to the manufacturers of start materials for R&D of new sunscreens (Flor, J. et al. “Protetores Solares” Química Nova, v. 30, 2007).

There are two classes of sunscreens: organic and inorganic, classified routinely and respectively as filters of chemical effect (chemical filters) and filters of physical effect (physical filters). Such classification presents only one commercial character and needs to be reevaluated. The processes of absorption and reflection of radiation are considered physical phenomena if there is no chemical reaction. This way, a UV radiation absorbing molecule should not be necessarily classified as chemical filter. The classification of organic and inorganic filters becomes more reasonable once the organic filters contain the organic compounds and the inorganic filters contain metallic oxides. Generally, the organic compounds protect the skin through the absorption of the radiation and the inorganic, by reflecting of the radiation. There are, currently, in the market organic filters that, besides absorbing, reflect the UV radiation. The Ciba Specialty Chemicals made available in the market the product Tinosob® M that, even being organic, presents the capacity of reflect and disperse the radiation, besides the capacity of absorb the UV radiation, behaving, this way, as a filter of also physical effect. It is important to notice that the phenomena of reflection and spreading depend on to the size of the particles of the inorganic filter, among other factors and not on the fact of the compound being organic or inorganic (Diffey, B. L., Grice, J. “The influence of sunscreen type on photoprotection” Br. J. Dermatol., 1997 apud Flor, J. et al. “Protetores Solares” Química Nova, v. 30, 2007).

The efficacy of a sunscreen is measured regarding its solar protection factor (SPF), which indicates how many times the sun exposition can be increased with the use of the sunscreen, without the risk of erythema. Considering, for example, the same geographical localizations, season of the year, climate conditions and period of the day, an individual of fair skin that can be exposed to the sun for 20 min without sunscreen, can be exposed to the sun for 300 min with an SPF=15 sunscreen, as 20×15=300. The higher the SPF, the higher the protection, which means, the higher the time in which the skin is protected from UVB radiation. It is important to notice that the SPF is defined regarding the UVB radiation which causes the erythemas. The SPF value is calculated through the equation below:

$$S P F = \frac{E M D ( skin with protection )}{E M D ( skin without protection )}$$

wherein EMD=erythematosus minimal dosage, which is, the minimum dosage needed to cause the erythema (Mansur, J. S. et al. “Correlação entre a determinação do fator de proteção solar em seres humanos e par espectrofotometria” Anais Bras. de Dermatologia, July-ago 1986 apud Flor, S. et al. “Protetores Solares” Química Nova, v. 30, 2007).

To make a solar filter available to the consumers, it is necessary that it is incorporated to a vehicle. To this solar filter/vehicle association is given the name of sunscreen or photoprotector. Some characteristics are required in order to make the sunscreens commercially available. Besides being chemically, photochemically and thermically inert, the sunscreens must present other characteristics, such as, for example, be non-toxic, non-sensitizing, non-irritating or non-mutagenic, non-volatile, present appropriate soluble characteristics, not be absorbed by the skin, not change its color, not cause stains on skin or clothes, be colorless, be compatible with the formulation and the storing material and be stable in the final product (Flor, J. et al. “Protetores Solares” Química Nova, v. 30, 2007).

In order to prepare a sunscreen, it is necessary the presence of two basic components: the active ingredients (organic and/or inorganic filters) and the vehicles. Many are the possible vehicles to be used in the preparation of sunscreens, involving from simple solutions to solutions with a more complex structure, such as emulsions (Flor, J. et al. “Protetores Solares” Química Nova, v. 30, 2007).

The main vehicles employed in photoprotective preparations can be (Flor, J. et al. “Protetores Solares” Química Nova, v. 30, 2007):

Hydro-alcoholic lotions—Composed mainly of water and alcohol, they are easy to spread in the skin and evaporate quickly. Its employment has been questioned due to the low obtained levels of protection. Besides that, the deleterious effect of ethylic alcohol on the skin has been also questioned.

Creams and emulsifier lotions—The emulsions constitute the best vehicle by far to the sunscreens. They consist of polar (hydrosoluble) as well as non-polar (liposoluble) components and can carry in their structure hydrosoluble filters as well as liposoluble filters, which is very healthy from protection's point of view. Such systems can be O/W (oil in water) or W/O (water in oil), characteristics that can also lead to preparations more or less protective. The emulsions W/O are more adequate for protection of the skin, however, they present high fatty or oleaginous characteristics, with consequent discomfort to the user. Because of that, the O/W emulsions constitute the most employed systems and guarantee adequate protection with a sensorial comfort to the user.

Gels—They are the vehicles obtained through a hydrophilic thickener. Independently from the origin of the thickener, whether natural (gums, alginates) or synthetic (polymers and acrylamide copolymers), the resulting gels generally do not offer the same levels of protection as the emulsions. Besides that, in order to keep the transparency characteristics of this group of preparations there is the necessity of the solar filters being hydrosoluble. As high levels of protection can only be achieved through the mixture of filters and as they are, in their majority, liposoluble, the obtaintion of transparent gels is an extremely delicate technical task and can involve the inclusion of not-always-desirable solvents, such as ethylic alcohol. In the preparation of photoprotective gels, the presence of inorganic filters must also be avoided. Even when in the form of microparticles, the inorganic filters render to the gel, in the best scenario, opaque aspect and, in most of the times, result in agglomerations visible to the consumer's eyes. The problem of these preparations is not only the esthetical appearance, but, fundamentally, the low resulting levels of protection. The presence of agglomerations in the sunscreen will lead to the formation of a non-homogeneous film in the whole extension of the skin, which greatly affects the level of protection.

Phenothiazines

Phenothiazines are compounds that present the molecular formula C₁₂H₉NS, the molecular weight of 199.28, CAS number 92-84-2, synonyms dibenzothiazine, dibenzo-p-thiazine, dibenzo-1,4-thiazine and 10H-phenothiazine, and the structural formula below (National Center for Biotechnology Information—NCBI: www.ncbi.nlm.nih.gov/):

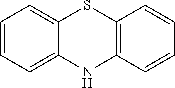

The phenothiazines are compounds that contain the thiazinic core composed by to and structure of three rings, wherein two benzenic rings are linked by a sulphur atom and a nitrogen atom.

According to the present invention, in order to obtain the photoprotective effect, there are no restrictions regarding the position of the substitutions.

On the other hand, as already disclosed in the state of the art, in the case of use as antipsychotic drugs, the substitutions in the phenothiazinic core are in the carbon 2 and the nitrogen 10.

Regarding the lateral substituent group in the position 10, these drugs can be subdivided in three subclasses (Baldessarini, R. J., Tarazi, F. I. “Drugs and the treatment of psychiatric disorders” Goodman and Gilman's the pharmacological basis of therapeutics, 10 ed, 1989; Wishart, D. S. et al. “DrugBank: a comprehensive resource for in silico drug discovery and exploration” Nucleic Acids Res. 2006):
aliphatic compounds, e.g. chlorpromazine, promazine, trimeprazine, propiomazine, triflupromazine, etopropazine and prometazine:

### Chlorpromazine

3-(2-chloro-10H-phenothiazin-10-yl)-N,N-dimethyl-propan-1-amine
C₁₇H₁₉ClN₂S
CAS 50-53-3

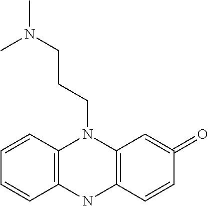

### Promazine

N,N-dimethyl-3-(10H-phenothiazin-10-yl)propan-1-amine
C₁₇H₂₀N₂S
CAS 58-40-2

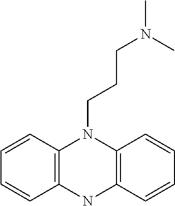

### Trimeprazine

N,N,2-trimethyl-3-phenothiazin-10-yl-propan-1-amine
C₁₈H₂₂N₂S
CAS 84-96-8

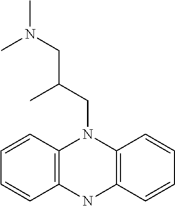

### Propiomazine

1-[10-(2-dimethylaminopropyl)-10H-phenothiazin-2-yl]propan-1-one
C₂₀H₂₄N₂OS
CAS 362-29-8

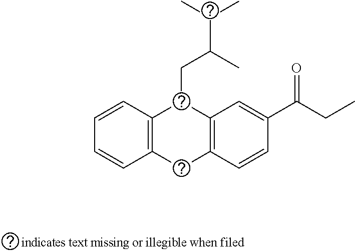

### Triflupromazine

N,N-dimethyl-3-[2-(trifluoromethyl)-10H-phenothiazin-10-yl]-propan-1-amine
C₁₈H₁₉F₃N₂S
CAS 146-54-3

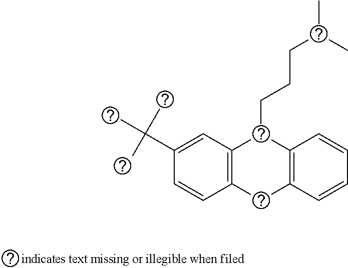

### Etopropazine

N,N-diethyl-1-(10H-phenothiazin-10-yl)propan-2-amine
C₁₉H₂₄N₂S
CAS 1094-08-2)

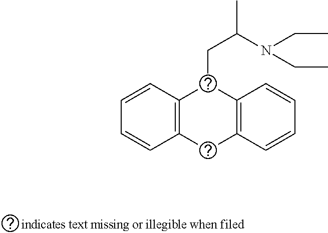

### Prometazine

N,N-dimethyl-1-(10H-phenothiazin-10-yl)propan-2-amine
C₁₇H₂₀N₂S
CAS 60-87-7

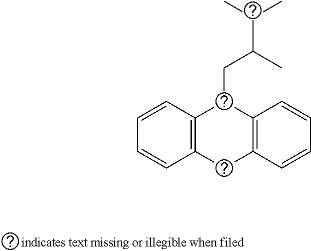

piperazinic compounds, e.g. trifluoperazine (TFP), fluphenazine (FP), prochlorperazine, perphenazine, thiethylperazine, acetophenazine and carphenazine:

### Trifluoperazine (TFP)

10-[3-(4-methylpiperazin-1-yl)propyl]-2-(trifluoromethyl)-10H-phenothiazine
C₂₁H₂₄F₃N₃S
CAS 117-89-5

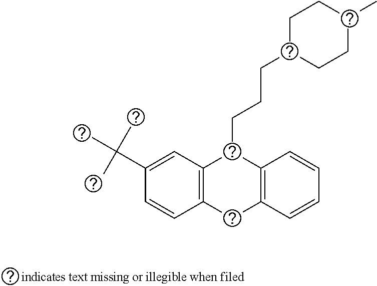

### Fluphenazine (FP)

2-[4-[3-[2-(trifluoromethyl)-10H-phenothiazin-10-yl]propyl]piperazin-1-yl]ethanol
C₂₂H₂₆F₃N₃OS
CAS 69-23-8

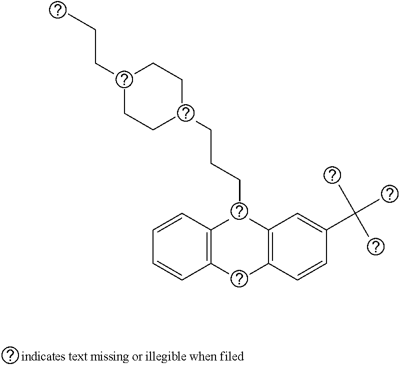

### Prochlorperazine

2-chloro-10-[3-(4-methylpiperazin-1-yl)propyl]-10H-phenothiazine
C₂₀H₂₄ClN₃S
CAS 58-38-8

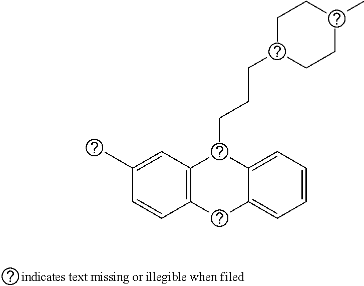

### Perphenazine

2-[4-[3-(2-chloro-10H-phenotiazin-10-yl)propyl]piperazin-1-yl]ethanol
C₂₁H₂₆ClN₃OS
CAS 58-39-9

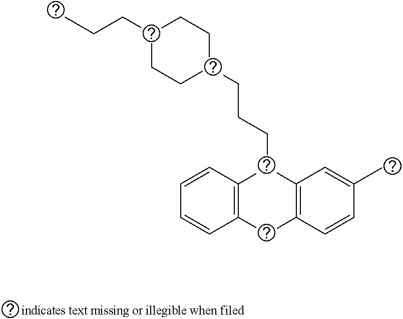

### Thiethylperazine

2-ethylsulfanyl-10-[3-(4-methylpiperazin-1-yl)propyl]-10H-phenothiazine
C₂₂H₂₉N₃S₂
CAS 1420-55-9

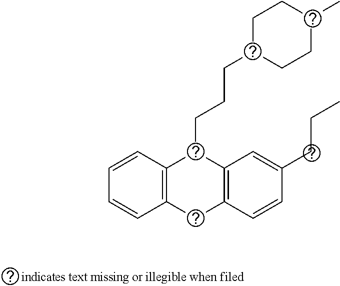

### Acetophenazine

1-[10-[3-[4-(2-hydroxietil)piperazin-1-yl]propyl]-10H-phenotiazin-3-yl]ethanone
C₂₃H₂₉N₃O₂S
CAS 2751-68-0

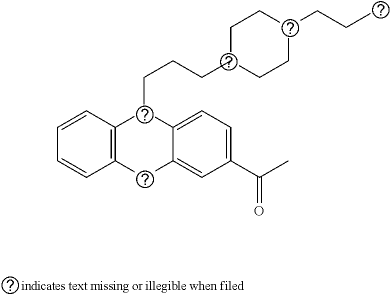

### Carphenazine

1-[10-[3-[4-(2-hydroxyethyl)piperazin-1-yl]propyl]phenothiazin-2-yl]propan-1-one
C₂₄H₃₁N₃O₂S
CAS 2622-30-2

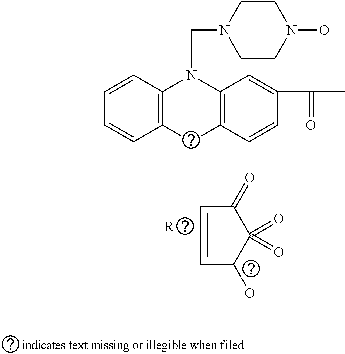

piperidinic compounds, e.g. thioridazine (TR), mesoridazine, mequitazine and metdilazine:

### Thioridazine (TR)

10-[2-(1-methyl-2-piperidyl)ethyl]-2-methylsulfanyl-phenothiazine
CAS 50-52-2
C₂₁H₂₆N₂S₂

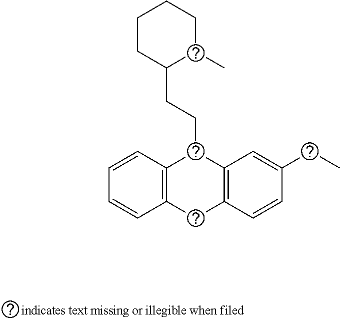

### Mesoridazine

10-[2-(1-methyl-2-piperidyl)ethyl]-2-methylsulfinyl-10H-phenotiazine
C₂₁H₂₆N₂OS₂
CAS 5588-33-0

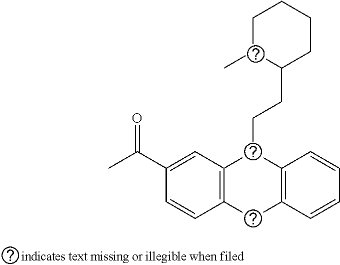

### Mequitazine

10-(4-azabicycle[2.2.2]oct-7-ylmethyl)phenothiazine
C₂₀H₂₂N₂S
CAS 29216-28-2

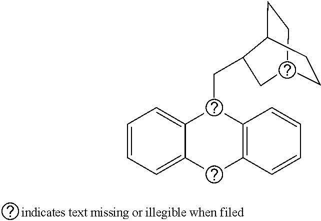

### Metdilazine

10-[(1-methylpirrolidin-3-yl)methyl]-10H-phenothiazine
C₁₈H₂₀N₂S
CAS 1982-37-2

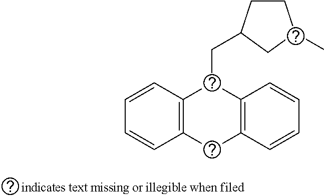

The phenothiazinic and derived compounds thereof has been the focus of several biological, chemical, physico-chemical and photochemical studies, due to their properties and applications. Particularly, the photochemical behavior of the phenothiazines has been of great interest, because compounds and compositions that consist of portions of phenothiazines can promote photosensitizing effect on people.

These photochemical properties originate from the structural likeness of these antipsychotic drugs with thiazinic colorants, such as methylene blue and the thionine, which photochemistry is already well known in the technique.

Studies have shown some effects of the phenothiazinic derivates in the excited state, such as damage to proteins and lipoperoxidation, using linoleic acid or erythrocytes' membranes. The authors describe that, in organic media, the decay of lower singlet excited state of the phenothiazinic derivates occurs by intersystem crossing to the lower triplet excited state, which is responsible for the generation singlet oxygen. Nevertheless, in aqueous media, the irradiation of these compounds seems to lead principally to the formation of the cationic radical of phenothiazine, which also presents pro-oxidant action, if it is not stabilized.

Therefore, the development of photoprotective formulations properly stabilized and that act in the whole UV spectrum is urgent.

### SUMMARY OF THE INVENTION

The present invention discloses processes of stabilization of cation radicals of one or more phenothiazinic compounds or derived compounds thereof which present the main structure:

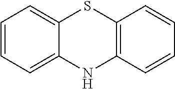

The present invention also discloses cosmeceutical formulations comprising one or more phenothiazinic compounds or derived compounds thereof which present the main structure:

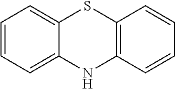

in combination with cosmeceutically acceptable excipients.

It is also embodiments of the present invention cosmeceutically formulations that still comprise cosmeceutical assistants, such as fragrance agents, coloring agents, antibacterial agents, insect repellent agents, vitaminic agents, antioxidant agents, preservative agents, emollient agents and others commonly employed in the art.

The present invention still discloses uses of one or more phenothiazinic compounds or derived compounds thereof which present the main structure:

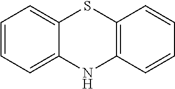

in the monomeric or polymeric form in the preparation of cosmeceutical formulations for the prevention of skin diseases and disturbances.

Other embodiments of the present invention relates to methods for prevention of skin diseases and disturbances comprising the administration of the formulations of the invention to an individual.

### BRIEF DESCRIPTION OF THE FIGURES

FIG. 1 presents the transmittance graphs A, B and C comprising the phenothiazine derived compounds of the present invention TR, TFP and FP, respectively, in which the presence of the drug is represented by the red line and its absence, by the black line.

FIG. 2 presents the effect of the concentration of the phenothiazine derived compounds of the present invention TR (graph A) and TFP (graph B), on the photooxidation of the model protein (methionine 80 of cytochrome c), measured by the degree of deviation to the blue of the Soret band.

FIG. 3 presents the effect of the pH of the media on the initial oxidation rate of methionine 80 of cytochrome c, in the presence and in the absence of TR 25 μM.

FIG. 4 present changes of spectrum of cytochrome c and of TR, promoted by irradiation of phenothiazine.

FIG. 5 presents the effect of the concentration and the aggregated state of TR in the alteration rate of Soret band. The curves show the effects of low concentrations of TR.

FIG. 6 presents the effect of the concentration of TR in the water superficial tension through experiments performed in one tensiometer De Noy, at room temperature, in deionized water.

FIG. 7 presents the area of UV light transmittance, in which the red line indicates the presence of the phenothiazinic core (PHT) and the black line indicates the absence of drugs.

### DETAILED DESCRIPTION OF THE INVENTION

The stabilization of the cation radical of the phenothiazines has been described only in extremely acidic media. However, it was developed mechanisms of stabilization of cation radical in mild conditions, such as buffered media slightly acidic (pH around 4.0) and pure water (pH around 6.0). The raise of the pH (up to around 8.0) leads to the creation of the neutral radical also less reactive. In these conditions, it was verified that drugs do not change their properties of UV light absorption and, at the same time, do not lead to the creation of reactive species which could, therefore, be used as sunscreen.

In the conditions in which the cation radical was stabilized, there is light absorption, without significant production of singlet oxygen, undesired reactive species which can be produced by the irradiation of known physical and chemical sunscreens, such as titanium oxide and the benzophenone. Other difficulty found, regarding the standard sunscreens, is a mechanism of visualization of its action. The stabilization of the cation radical from diverse phenothiazines transforms them into reversibly colored substances. When the exposition to light ceases, the color decays, but reappears under new exposition. In these conditions, the photodegradation is low and should not be accounted for, and data are obtained about the maintenance of stability for up to 25 days of daily sessions of irradiation.

The present invention, therefore, relates to processes of stabilization of cation radicals of one or more phenothiazinic compounds or derived compounds thereof, which presents the main structure:

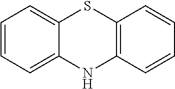

In one embodiment of the present invention, the said one or more phenothiazinic compounds or derived compounds thereof present the Formula I:

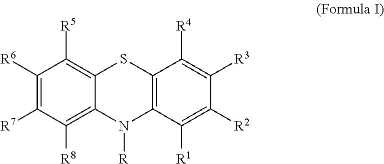

wherein R, R¹, R², R³, R⁴, R⁵, R⁶, R⁷ and R⁸ are independently selected from the group consisting of hydrogen, hydroxyl, halogen, alkyl, amine, amino, ketone, piperazine, trifluoromethyl, sulphanyl, piperidine, sulphynyl, azabicycle, pirrolidine, alkoxi, alkenyl, alkinyl, sulphidryl, amide, nitro, ciano and acyl wherein the cited substituents are and/or present substituted substituents or non-substituted, saturated or unsaturated and/or cyclic or of open chain.

In another embodiment of the present invention, the said one or more phenothiazinic compounds or derived compounds thereof are selected from the group consisting of phenothiazine, chlorpromazine, promazine, trimeprazine, propiomazine, triflupromazine, etopropazine, prometazine, trifluoperazine (TFP), fluphenazine (FP), prochlorperazine, perphenazine, thiethylperazine, acetophenazine, carphenazine, thioridazine (TR), mesoridazine, mequitazine and metdilazine. Preferentially, the said one or more phenothiazinic compounds or derived compounds thereof are trifluoperazine (TFP), fluphenazine (FP) and thioridazine (TR).

The said processes comprise to initially mix the start compounds benzenethiols or substituted anilines or not to the anilines or benzenethiols reagents, and sulphur, iodine and solvents, under reaction conditions of high temperature.

The cation radical is formed photochemically by UV irradiation or chemically by the use of oxidants or peroxidases enzymes.

In the aggregated state and at acidic pH, the stability can last for hours if the sample is kept in low temperatures and/or under irradiation.

The said one or more phenothiazinic compounds or derived compounds thereof used in the present invention can be chosen among the thioridazine (TR) or the fluphenazine (FP). The said phenothiazines TR and FP are found present in the reaction media under concentration ranging from around 5 μM (TR and FP) to around 2.5 mM (TR) and around 100 □M (FP). In a particular embodiment, the concentrations range from around 200 μM to around 2.5 mM (TR) and from around 50 □M to around 100 □M (FP). In a more particular embodiment, the concentrations range from around 400 μM to around 2.5 mM (TR) and from around 75 □M to around 100□M (FP). In a still more particular embodiment, the concentrations one or more phenothiazinic compounds or derived compounds thereof are around 2.5 mM for TR and 100 □M for FP.

Besides the abovementioned phenothiazinic compounds, the processes of the present invention can use other(s) phenothiazinic compound(s) or derived compounds thereof, in concentrations ranging from around 5 μM to around 2.5 mM.

The reaction media of the said processes present a pH ranging from around 4.0 to around 8.0. In a particular embodiment, the said pH range can be from around 5.0 to around 7.0. In a more particular embodiment, the pH is around 6.0.

The present invention relates to, still, the cosmeceutical formulations comprising one or more phenothiazinic compounds or derived compounds thereof, which present the main structure:

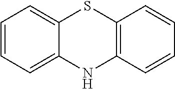

in the monomeric or polymeric form, in combination with cosmeceutically acceptable excipients.

In a particular embodiment, the said one or more phenothiazinic compounds or derived compounds thereof, present in the formulations of the invention, present the Formula I:

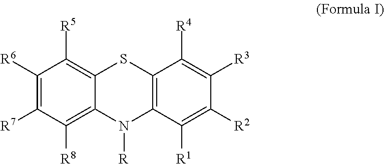

wherein R, R¹, R², R³, R⁴, R⁵, R⁶, R⁷ and R⁸ are independently selected from the group consisting of hydrogen, hydroxyl, halogen, alkyl, amine, amino, ketone, piperazine, trifluoromethyl, sulphanyl, piperidine, sulphynyl, azabicycle, pirrolidine, alkoxi, alkenyl, alkinyl, sulphidryl, amide, nitro, ciano and acyl wherein the cited substituents are and/or present substituted substituents or non-substituted, saturated or unsaturated and/or cyclic or of open chain.

In a more particular embodiment, the abovementioned one or more phenothiazinic compounds or derived compounds thereof are selected from the group consisting of phenothiazine, chlorpromazine, promazine, trimeprazine, propiomazine, triflupromazine, etopropazine, prometazine, trifluoperazine (TFP), fluphenazine (FP), prochlorperazine, perphenazine, thiethylperazine, acetophenazine, carphenazine, thioridazine (TR), mesoridazine, mequitazine and metdilazine. Preferentially, the said one or more phenothiazinic compounds or derived compounds thereof are trifluoperazine (TFP), fluphenazine (FP) and thioridazine (TR).

It is a distinction of the present invention that the said one or more phenothiazinic compounds or derived compounds thereof are found in the form of stabilized cation radicals. It is another distinction that the said one or more phenothiazinic compounds or derived compounds thereof are found in the monomeric and polymeric form, particularly in the form of pre-micellic aggregates and/or micelles.

The said one or more phenothiazinic compounds or derived compounds thereof used in the present invention can be chosen among the thioridazine (TR) or the fluphenazine (FP). The said phenothiazines TR and FP are found present in the cosmeceutical formulations in concentration ranging from around 5 μM (TR and FP) to around 2.5 mM (TR) and around 100□M (FP). In a particular embodiment, the concentrations range from around 200 μM to around 2.5 mM (TR) and from around 50□M to around 100□M (FP). In a more particular embodiment, the concentrations range from around 400 μM to around 2.5 mM (TR) and from around 75□M to around 100□M (FP). In a still more particular embodiment, the concentrations one or more to phenothiazinic compounds or derived compounds thereof are around 2.5 mM for TR and 100 □M for FP.

Besides the abovementioned phenothiazinic compounds, the processes of the present invention can use other(s) phenothiazinic compound(s) or derived compounds thereof, in the cosmeceutical formulations, in concentrations ranging from around 5 μM to around 2.5 mM.

The cosmeceutical formulations of the invention present a pH ranging from around 4.0 to around 8.0. In a particular embodiment, the said pH range can be from around 5.0 to around 7.0. In a more particular embodiment, the pH is around 6.0.

The said cosmeceutically acceptable excipients present in the formulations of the invention are selected from the group consisting of vehicle, agglutinants, desintegrants, binders, lubricants, surfactants, solubilizers, suspending agents, thickeners, diluents, solvents, emulsifiers, stabilizers, preservatives, colorants, seasonings, combinations thereof and others commonly employed in the art.

Examples of vehicles which can be employed in the present invention are, but not limited to: water, aqueous solution, vegetable oils, mineral oils, combinations thereof and others commonly known to those skilled in the technique.

Examples of agglutinants that can be employed in the present invention are, but not limited to: methylcellulose, carboxymethylcellulose, gum arabic, gelatin, glucose, dextran, povidone, amide, combination thereof and others commonly known to those skilled in the technique.

Examples of desintegrants that can be employed in the present invention are, but not limited to: alginic acid, alginates, carboxymethylcellulose, amide, combination thereof and others commonly known to those skilled in the technique.

Examples of binders that can be employed in the present invention are, but not limited to: gelatin, carrageen, combination thereof and others commonly known to those skilled in the technique.

Examples of lubricants that can be employed in the present invention are, but not limited to: estearic acid, estereates, mineral oils, combination thereof and others commonly known to those skilled in the technique.

Examples of surfactants that can be employed in the present invention are, but not limited to: polysorbates, benzalconic chloride, sorbitan monopalmitate, sodium lauryl ether sulfate, combination thereof and others commonly known to those skilled in the technique.

Examples of solubilizers that can be employed in the present invention are, but not limited to: Cremophor®, caprylic glycoside, PPG-5 Ceteth-20, combination thereof and others commonly known to those skilled in the technique.

Examples of solubilizers that can be employed in the present invention are, but not limited to: polyvinilpirrolidone, colloidal silicon, polysaccharides, combination thereof and others commonly known to those skilled in the technique.

Examples of thickeners that can be employed in the present invention are, but not limited to: coconut fatty acid diethanolamide, myristic acid, lauric acid, oleic acid, salts, alginates, carboxymethylcellulose, methylcellulose, fatty acid alkanolamides, silicas, combination thereof and others commonly known to those skilled in the technique.

Examples of diluents that can be employed in the present invention are, but not limited to: caulim, lactose, mannitol, microcrystalline cellulose, sorbitol, calcium carbonate, combination thereof and others commonly known to those skilled in the technique.

Examples of solvents that can be employed in the present invention are, but not limited to: acetone, polyethylene glycol (PEG), alcohols, vegetable oils, glycerin, oleic acid, mineral oils, water, combination thereof and others commonly known to those skilled in the technique.

Examples of emulsifiers that can be employed in the present invention are, but not limited to: cetomacrogol, cetylic acid, glyceryl monostearate, sorbitane monooleate, combination thereof and others commonly known to those skilled in the technique.

Examples of stabilizers that can be employed in the present invention are, but not limited to: coconut fatty acid diethanolamide, formaldehyde, combination thereof and others commonly known to those skilled in the technique.

Examples of preservatives that can be employed in the present invention are, but not limited to: parabens, benzoic acid, sodium benzoate, sodium propionate, benzalconic chloride, benzylic alcohol, phenols, butylhydroxytoluene (BHT), butylhydroxyanisol (BHA), Nipagin® (methylparaben), Nipazol® (propylparaben), combination thereof and others commonly known to those skilled in the technique.

Examples of colorants that can be employed in the present invention are, but not limited to: caramel, ferric oxide, D&C Orange No. 5, FD&C Yellow No. 6, titanium dioxide, combination thereof and others commonly known to those skilled in the technique.

Examples of seasonings that can be employed in the present invention are, but not limited to: vegetable oils or fragrances, menthol, vanilla, aspartame, dextrose, mannitol, combination thereof and others commonly known to those skilled in the technique.

The cosmeceutical formulations of the present invention can still comprise cosmeceutical assistants selected from the group consisting of solar protection agents, fragrance agents, antibacterial agents, insect repellent agents, vitaminic agents, antioxidant agents, emollient agents, pH correction agents, combination thereof and others.

Examples of solar protection agents that can be employed in the present invention are, but not limited to: octocrylene, avobenzone, oxybenzone (benzophenone-3), Tinosorb® S, octyl paramethoxycinnamate, octyl salicylate, methylbenzylidene camphor, octyl triazone, cinoxate, homosalate, octyl methoxycinnamate, Padimato® O, phenylbenzimidazole sulfonic acid, sulisobenzone, TEA-salicylate, oxibenzone, dioxybenzone, sulisobenzone and mixes thereof; ethylhexyl methoxycinnamate, aminobenzoic acid, phenylbenzimidazole sulfonic acid, sulisobenzone, digalloyl trioleate, diethanolamine methoxycinnamate, dioxybenzone, ethyl-4-bis(hydroxypropyl)aminobenzoate, 2-ethylhexyl 2-cyano-3,3-diphenylacrylate, homosalate, glyceryl aminobenzoate, menthyl anthranilate, octocrilene, ethylhexyl salicylate, Padimato® A, ethylhexyl methoxycinnamate (Uvinul® MC 80), aminobenzoic acid, phenylbenzimidazole sulfonic acid, sulisobenzone, combination thereof and others commonly known to those skilled in the technique.

Examples of fragrance agents that can be employed in the present invention are, but not limited to: vegetable oils or fragrances, menthol, vanilla, combination thereof and others commonly known to those skilled in the technique.

Examples of antimicrobial agents that can be employed in the present invention are, but not limited to: parabens, benzoic acid, sodium benzoate, sodium propionate, benzalconic chloride, benzylic alcohol, phenols, butylhydroxytoluene (BHT), butylhydroxyanisol (BHA), Nipagin®, Nipazol®, combination thereof and others commonly known to those skilled in the technique.

Examples of insect repellent agents that can be employed in the present invention are, but not limited to: citronellal, geraniol, combination thereof and others commonly known to those skilled in the technique.

Examples of vitaminic agents that can be employed in the present invention are, but not limited to: vitamin A, vitamin E, vitamin C, combination thereof and others commonly known to those skilled in the technique.

Examples of antioxidant agents that can be employed in the present invention are, but not limited to: ascorbic acid, monothioglycerol, propylgalate, sodium ascorbate, sodium bisulfite, combination thereof and others commonly known to those skilled in the technique.

Examples of emollient agents that can be employed in the present invention are, but not limited to: Luvitol®, triglycerides, vegetable oils, glycosaminoglycan, hydrolyzed protein, tocopherol acetate, pantothenic, combination thereof and others commonly known to those skilled in the technique.

Examples of pH correction agents that can be employed in the present invention are, but not limited to: citric acid, acetic acid, nitric acid, ammonia solution, sodium carbonate, sodium hydroxide, triethanolamine, potassium metaphosphate, sodium acetate, combination thereof and others commonly known to those skilled in the technique.

The said one or more phenothiazinic compounds or derived compounds thereof present in the formulation of the invention are comprised in dispersions, emulsions, pastes, powders, solutions, creams, colloids, gels, oils, macrocapsules, microcapsules, nanocapsules, macrospheres, microspheres, nanospheres, liposomes, oleosomes, chylomicrons, macroparticles, microparticles, nanoparticles, macrosponges, microsponges, nanosponges and others, or are found adsorbed in organic polymeric powders, talcs, bentonites and other organic or inorganic supports.

The cosmeceutical formulations of the present invention are comprised in macrocapsules, microcapsules, nanocapsules, macrospheres, microspheres, nanospheres, liposomes, oleosomes, chylomicrons, macroparticles, microparticles, nanoparticles, macrosponges, microsponges, nanosponges and others, or are found adsorbed in organic polymeric powders, talcs, bentonites and other organic or inorganic supports, or are found in the form of dispersions, emulsions, pastes, powders, solutions, creams, colloids, serum, gels, oils, cream-gel, oil-gel, lotions, bases, ointments, unguents, milks, suspensions, foam, sprays, roll-on, sticks, lipsticks, patches and others.

Other embodiments of the present invention are the uses of one or more phenothiazinic compounds or derived compounds thereof, which present the main structure:

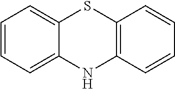

in the preparation of the cosmeceutical formulations to prevent skin diseases and disturbances.

In a particular embodiment, the said one or more phenothiazinic compounds or derived compounds thereof used in the above in the preparation of formulations of the invention, present the Formula I:

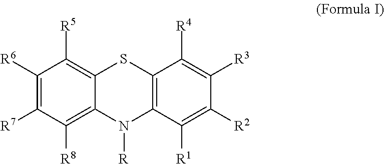

wherein R, R¹, R², R³, R⁴, R⁵, R⁶, R⁷ and R⁸ are independently selected from the group consisting of hydrogen, hydroxyl, halogen, alkyl, amine, amino, ketone, piperazine, trifluoromethyl, sulphynyl, piperidine, sulphynyl, azabicycle, pirrolidine, alkoxi, alkenyl, alkinyl, sulphidryl, amide, nitro, ciano and acyl wherein the cited substituents are and/or present substituted substituents or non-substituted, saturated or unsaturated and/or cyclic or of open chain.

In a more particular embodiment, the said one or more phenothiazinic compounds or derived compounds thereof are selected from the group consisting of phenothiazine, chlorpromazine, promazine, trimeprazine, propiomazine, triflupromazine, etopropazine, prometazine, trifluoperazine (TFP), fluphenazine (FP), prochlorperazine, perphenazine, thiethylperazine, acetophenazine, carphenazine, thioridazine (TR), mesoridazine, mequitazine and metdilazine. Preferentially, the said one or more phenothiazinic compounds or derived compounds thereof are trifluoperazine (TFP), fluphenazine (FP) and thioridazine (TR).

It is a distinction of the present invention that the said one or more phenothiazinic compounds or derived compounds thereof are found in the form of stabilized cation radicals. It is another distinction that the said one or more phenothiazinic compounds or derived compounds thereof are found in the monomeric and polymeric form, particularly in the form of pre-micellic aggregates and/or micelles.

Other embodiments of the present invention are the methods of prevention of skin diseases and disturbances that consist in the administration of cosmeceutical formulations of the invention to an individual. In a particular embodiment, the said administration is topic administration and the said skin disturbances are selected from the group consisting of aging, wrinkles, skin rashes, drying, oxidation, burns, erythemas, dermatoses, dermatites, cancers and others.

By means of skin application of a prophylactically or therapeutically efficient quantity of the cosmeceutically formulations of the present invention, before and/or during exposition of the skin to the sun e/or to systems of artificial tanning, the occurrence of the said skin diseases/disturbances is prevented and avoided.

Among the benefits brought by the present invention, it can be highlighted, but not limited to:
(i) broad range of UV light absorption of A, B and C of the electromagnetic radiation spectrum;
(ii) stability maintained for long periods of irradiation, which is a time period far superior to the expected to a day of sun exposure, in the hours of most incidence of UV rays;
(iii) low quantic rate of generation of singlet oxygen by means of mechanisms of energy transfer;
(iv) low reactivity, allowing association to other components of the formulation;
(v) low relative cost for use and obtaintion of different chemical derivates that could increase its efficacy; and
(vi) high efficacy of photoprotection, in dermatological pH (around 5.5).

Some illustrative examples of the cosmeceutical formulations enclosed by the present invention are presented below, as well as the respective obtained results of the new formulations described herein. The Examples listed below are provided by way of illustration and not by way of limitation.

### EXAMPLES

### Example 1

In order to analyze the transmittance of UV light in the wavelength of 320 to 360 nm, solutions of 2.0 mg/cm² of the phenothiazines TR, TFP and FP were used in a quartz plate.

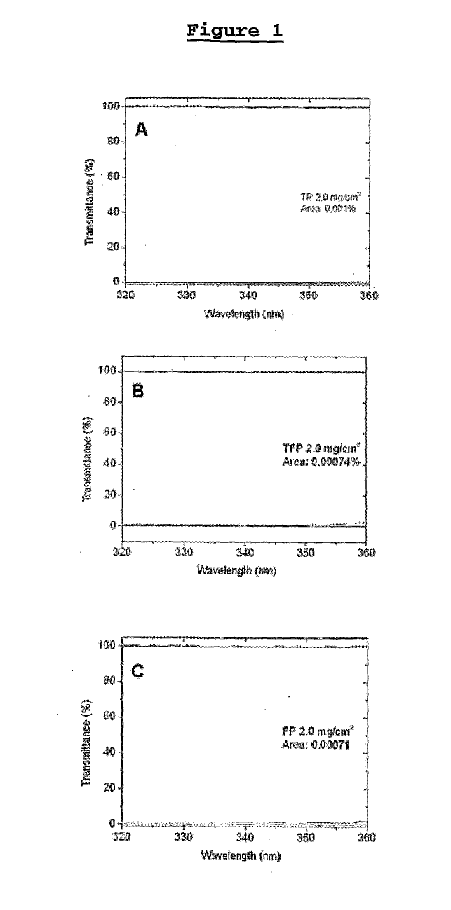

As it can be observed, in these conditions, TR, TFP and FP block the passage of almost 100% of UV light incidence (FIG. 1). This evaluation was obtained based on the Australian Method (Perassinoto, N. L., Journal In Comesto, VII Ed., 2006), which was elected the most adequate, because it considers the ε of UVA region of absorption. The other methods are based in the 320 and 360 nm absorption ratio and can generate false negative results. According to the Australian Method, it will be a good sunscreen the compound that blocks 90% of UVA light incidence. In the case of the said cation radicals of the phenothiazines of the present invention, the blockage was of 99.99%.

### Example 2

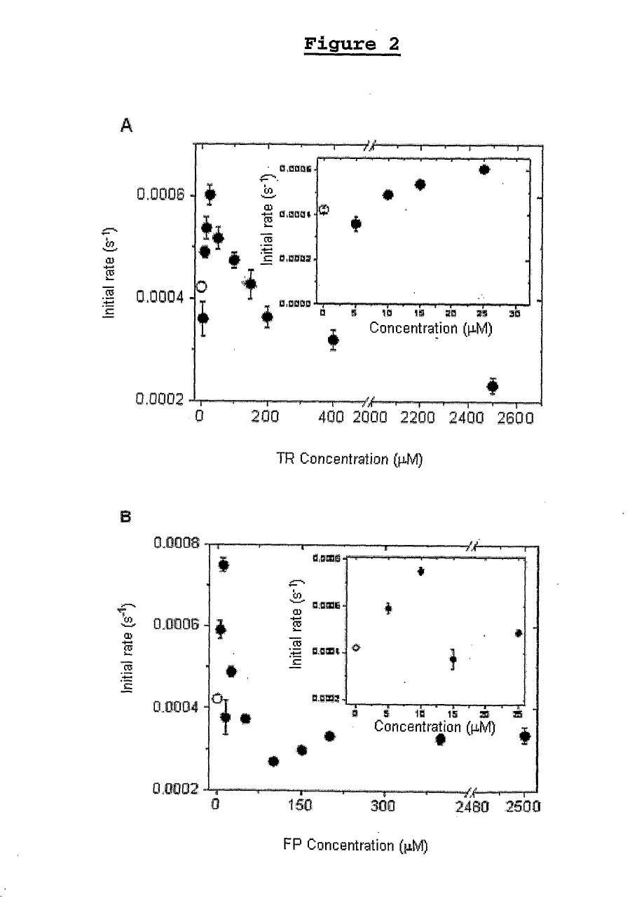

It was analyzed the effect of the concentration of the phenothiazine derived compounds of the present invention TR (graph A, FIG. 2) and TFP (graph B, FIG. 2), on the photooxidation of the model protein (methionine 80 of cytochrome c), measured by the degree of deviation to the blue of the Soret band.

The white circle, in A and B, represents the damage caused to the protein after two hours of irradiation under UV light at 254 nm, at temperature of 25° C., at pH 4. In very low concentrations, the drug TR (graph A) leads to slight protection, meaning that there was a disequilibrium between the generation of cation radical and the quantity of absorbed light, favoring the light absorption, which leads to protection.

The increase of the drug concentration exacerbated the damage up to the concentration of 25 μM for TR (graph A) and of 10 μM for FP (graph B), suggesting that the protection that would be promoted by the absorption of light was supplanted by the increase of the cation radical quantity generated. However, from these concentration values up, the damage caused to the protein decreases with the increase of the drug concentration and begins to protect the protein from the damage caused by the UV light.

### Example 3

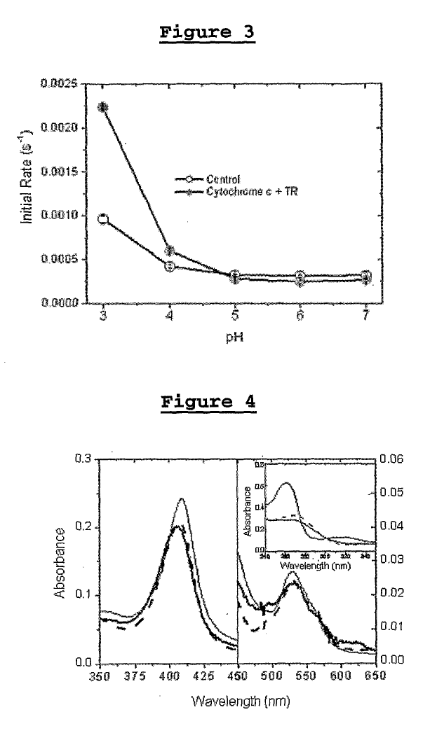

The effect of the media pH on the initial velocity of oxidation of methionine 80 of cytochrome c was analyzed at an UV irradiation of 254 nm, during 120 min, in the absence and in the presence of the stabilized cation radicals of TR, TFP and FP (FIG. 3). In the presence of the stabilized cation radicals of TR, TFP and FP, de concentration range of 5 to 2500 μM for pH 4.0 and the concentration range of 25 to 2500 μM for the pH range of 3.0 to 7.0 were used.

In the absence of the stabilized cation radicals of the phenothiazines, at a pH=4.0, the irradiation promoted the dislocation of the Soret band from 409 to 406 nm, with an initial rate of dislocation to the blue of 0.42 ms⁻¹.

In the presence of the stabilized cation radicals of the phenothiazines in concentrations from 5 to 25 μM, at a pH=4.0, the irradiation increased and accelerated the damage to the cytochrome c (Soret band of 405 nm and initial rate of dislocation to the blue of 0.6 ms⁻¹).

In the presence of the stabilized cation radicals of phenothiazines in concentrations from 25 to 2500 μM, at pH=4.0, the irradiation protected the cytochrome c from the effects of UV light (Soret band of 407 nm and initial rate of dislocation to the blue of 0.23 ms⁻¹, with TR 2500 μM).

Particularly with stabilized cation radicals of TR 25 μM, it was observed that this concentration progressively increased the damage to cytochrome c in the pH range from 5.0 to 3.0, while it slightly protected the cytochrome c in the pH range from 5.0 to 7.0.

It is observed in FIG. 3 that, even in the concentration of maximum damage capacity, there is a protection tendency in pH above 4.8. Taking into consideration that 5.5 is the skin pH, high levels of protection can be achieved combining high concentrations (about 500 μM to about 2500 μM) of the drug with dermatologic pH.

### Example 4

Samples containing cytochrome c 3 μM and TR 25 μM were irradiated at 254 nm, in phosphate buffer 5 mM, pH=4.0, at 35° C., in a cuvette, at 4 cm from a UV lamp of 4 W. The spectrum of cytochrome c and of phenothiazine were obtained before the irradiation (fine solid line of FIG. 4) and after 60 and 120 min of irradiation (dotted line and thick solid line, respectively, of FIG. 4). The spectrum shown in the curves corresponds to the overlapping spectrum changes of the phenothiazines detected during the same indicated time points.

The cytochrome c spectrum, in the presence of the phenothiazine, before the irradiation (time zero), was typical of the Fe (III) low spin state of the native cytochrome c (thick solid line of FIG. 4). This result indicated that the phenothiazine did not induced significant alteration in the structure of cytochrome c and in the spin states of heme's iron.

After irradiation, at pH=4.0, two phenomena occurred simultaneously: dislocation to the blue and discoloration of the Soret band of cytochrome c and the conversion of the phenothiazine in the corresponding sulfoxide derivates (FIG. 4). Similar results were obtained with FP and TFP.

Laboratory data demonstrated that the phenothiazines TR, FP and TFP have formed pre-micellic aggregates and micelles that stabilize their cation radicals derivates, as shown below. The said cationic radicals exhibit absorption bands at 615 nm (TR) and 520 nm (FP and TFP).

The phenothiazines TR, FP and TFP (Sigma Chemical Co.) were obtained and aqueous solutions were prepared with deionized water. The solution of aggregated phenothiazines (pre-micellic and/or micelles) were prepared by the dissolution of surfactants in appropriated buffer, under agitation, at 37° C.

Samples of monomeric and aggregated forms of the phenothiazines were submitted to a UV lamp (4 W) at 254 nm or 365 nm, during 20 minutes, in buffered acidic media (pH from around 4.0 to around 6.0), for intervals of 1, 5, 10, 15, 20 and 25 days. Mass spectrometry of the aggregated forms of the cation radicals of phenothiazines (>100 μM) demonstrated that significant concentrations of the oxidized derivates were present only in the samples irradiated during 20 and 25 days. By contrast, the monomeric forms of the cation radicals of phenothiazines were totally converted into oxidized forms after 20 min of irradiation. Therefore, it can be concluded that excited states of the triplets of the aggregated forms of the cation radicals of phenothiazines are able to form stabilized cation radicals, possibly due to de packing of the phenyl thiazin portions. This result demonstrated the stabilization of the cation radicals of phenothiazines in their aggregated states.

### Example 5

The experiment described in the Example 4 was repeated for different concentrations of phenothiazines. The initial rate of dislocation to the blue of the Soret band was defined regarding the concentration of phenothiazine.

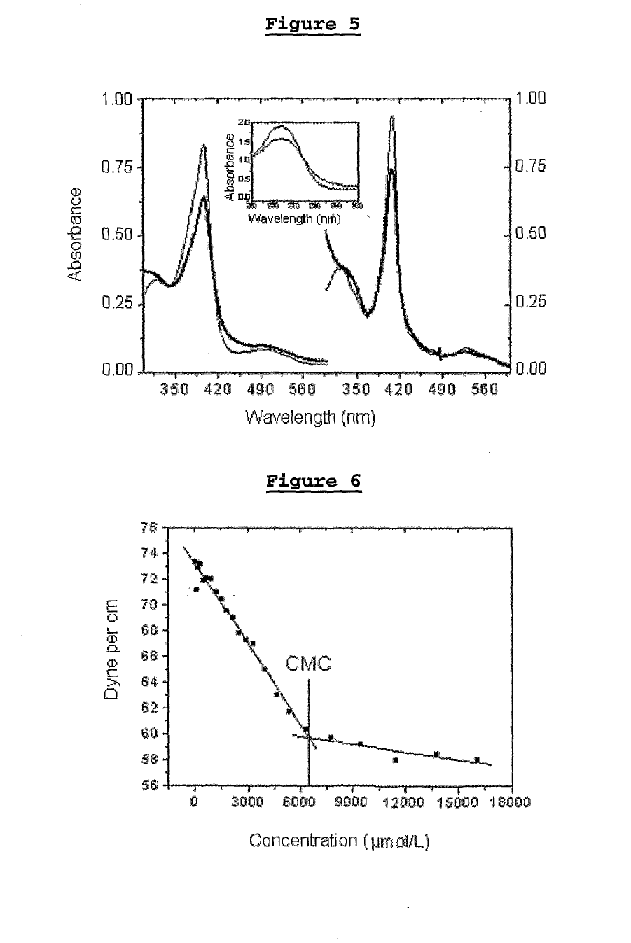

The irradiation of the cytochrome c was performed during 120 min, pH=4.0 and under UV lamp of 4 W, which led to the dislocation to the blue of the Soret band from 409 to 406 nm, with initial rate of 0.42 ms⁻¹ (open circle, FIG. 5).

In the presence of TR 5 μM, it was observed a discrete reduction in the dislocation rate to the blue. Above the concentration of 5 μM, up to 25 μM, TR increase and accelerated the damage to cytochrome c (Soret band of 405 nm and initial rate of dislocation to the blue of 0.6 ms⁻¹, obtained in the presence of TR 25 μM—FIG. 5).

Above the concentration of 25 μM, the concentration increase of TR progressively protected the cytochrome c from the effects of UV light (Soret band of 407 nm and initial rate of dislocation to the blue of 0.23 ms⁻¹, obtained in the presence of TR 2500 μM).

Therefore, in high concentrations, TR reduced almost around 50% of the initial rate of dislocation to the blue of the Soret band of cytochrome c, promoted by UV irradiation.

### Example 6

Irradiation of the cytochrome c 3 μM was performed, for a pH variation range from 3.0 to 7.0. In this pH range, the phenothiazine remained predominately protonated due to its pKa of 8.1. In the experimental conditions, the cytochrome c exhibit the Soret band of 409 nm, indicating the native low spin state, in a pH range from 4.0 to 7.0. Below pH=4.0, in the experimental conditions and before irradiation, the cytochrome c exhibit the Soret band of 406 nm, indicating transition to a less structured state. In the said pH range, the initial rate of dislocation to the blue of the Soret band was determined, after 120 min of UV irradiation, in the presence and in the absence of TR (FIG. 6).

In the pH range from 5.0 to 7.0, TR 25 μM promoted slight reduction of the rate of dislocation to the blue of the Soret band. At pH=4.0, TR increased the initial rate of dislocation to the blue in almost 50%. At pH=3.0, the said increase reached 250%. This result indicated that, in the pH range between 5.0 and 7.0, the presence of TR prevented UV light damage of the cytochrome c due to light absorption. However, the UV light absorption generated the cation radical of TR, which is also capable of promoting oxidative damage in the methionine 80 of cytochrome c.

Therefore, the damage to cytochrome c, promoted by UV light, was substituted by the damage promoted by the cation radical of TR, with similar intensity. Below pH=5.0, an increase of the yield of cation radical of TR that favors the damage was expected, besides the alterations promoted by the pH in the cytochrome c structure.

It is particularly interesting to note that the transition of cytochrome c, from the native form to the “fused globule” structure, exacerbated more drastically the oxidation of the methionine 80, promoted by the phenothiazine promoted by UV light.

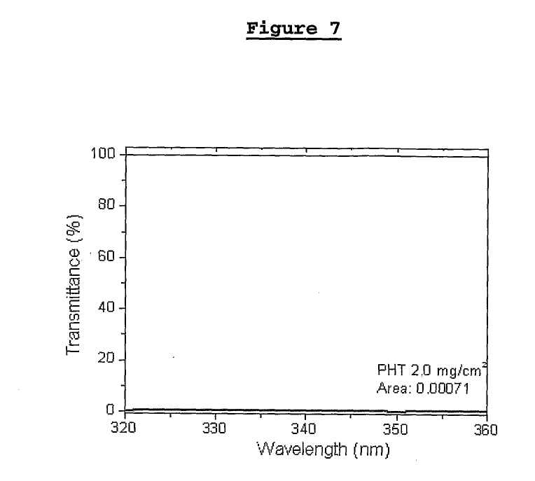

The irradiation of cytochrome c, at a pH=8.0, in the presence of phenothiazines, resulted in a complete protection of the protein from the oxidative damage promoted by UV light. Still, small quantities of the reduced form of the protein were detected (FIG. 7).

Above pH=8.0, the phenothiazines exhibited low solubility, which makes rather difficult to perform any investigations under these conditions.

### Example 7

### Cosmeceutical Formulation Obtaintion

The employment of the phenothiazines in the processes and formulations of the invention, for the formation and stabilization of the corresponding cation radicals, it will be made with raw material compounds previously synthesized and acquired from commercial suppliers.

In the case of new structures, these should be synthesized according to the information disclosed herein, preferentially with the employment of substituted anilines as start compounds.

The cream Lanette® N (auto emulsionant anionic wax of polyoxyethylene alcohol with sodium sulfate alkyl) was pharmocotechnically prepared, weighting separately the aqueous phase components (glycerin, Nipagim®, EDTA dissodic and distilled water) and the oleaginous phase (Lanette® N, silicon oil, Cetiol® V, Nipazol and BHT), in a beacker. It was heated to the temperature of 70° C. The aqueous phase containing the stabilized cation radical of phenothiazine was poured over the oleaginous to one, under constant agitation and cooling, until the emulsion was formed.

### Example 8

A solution of 2.0 mg of drug/cm² was submitted to an analytical assay in a quartz plate, in the wavelength range from 320 to 360 nm, in order to verify the area of UV light transmittance (FIG. 7).

In these conditions, the phenothiazinic core (PHT) blocked the passage of almost 100% of UV light incidence.

This evaluation was performed based in the Australian Method (Perassinoto, N. L.; Journal In Comesto; VII edition; p. 06; 2006), which was elected the most adequate, because it considers the ε of UVA region of absorption. The other methods are based in the 360 and 320 nm absorption ratio and can generate false negative results. According to the Australian Method, it will be a good sunscreen the compound that blocks 90% of UVA light incidence.

The said analytical assay made it possible to conclude that the phenothiazinic core, either substituted or non-substituted, is sufficient and highly satisfactory to confer the desired photoprotection according to the present invention.

1-28. (canceled)

29. Processes of stabilization of cation radicals of one or more phenothiazinic compounds or derived compounds thereof, wherein said compounds present the main structure: 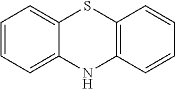
wherein initially the start compounds benzenethiols and substituted or not anilines, are mixed with the anilines or benzenethiols reagents, and sulphur, iodine and solvents, under reaction conditions of high temperatures, then the phenothiazinic compounds are transformed into cation radicals photochemically or chemically, followed by stabilization thereof under mild conditions, in the aggregated state or micelles, without significant production of the singlet oxygen and photodegradation of the compounds.

30. Processes according to claim 29, wherein said one or more phenothiazinic compounds or derived compounds thereof present the Formula I: 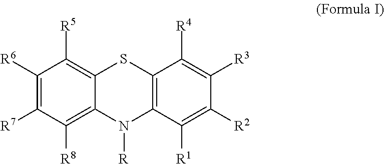
wherein R, R¹, R², R³, R⁴, R⁵, R⁶, R⁷ and R⁸ are independently selected from the group consisting of hydrogen, hydroxyl, halogen, alkyl, amine, amino, ketone, piperazine, trifluoromethyl, sulphanyl, piperidine, sulphynyl, azabicycle, pirrolidine, alkoxi, alkenyl, alkinyl, sulphidryl, amide, nitro, ciano and acyl wherein the cited substituents are and/or present substituted substituents or non-substituted, saturated or unsaturated and/or cyclic or of open chain.

31. Processes according to claim 29 or 30, wherein said one or more phenothiazinic compounds or derived compounds thereof are selected from the group consisting of phenothiazine, chlorpromazine, promazine, trimeparzine, propiomazine, triflupromazine, etopropazine, prometazine, trifluoperazine (TFP), fluphenazine (FP), prochlorperazine, perphenazine, thiethylperazine, acetophenazine, carphenazine, thioridazine (TR), mesoridazine, mequitazine and metdilazine.

32. Processes according to claim 31, wherein said photochemical or chemical process consists of UV irradiation or use of oxidants or peroxidases enzymes.

33. Processes according to claim 32, wherein said mild stabilization conditions relates to a media which pH varies from 4.0 to 8.0.

34. Processes according to claim 33, wherein said one or more phenothiazinic compounds or derived compounds thereof are present in concentration raging from around 5 μM to around 2.5 mM.

35. Cosmeceutical formulations, wherein said cosmeceutical formulations comprise one or more phenothiazinic compounds or derived compounds thereof, wherein said compounds present the main structure: 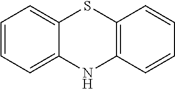
in combination with cosmeceutically acceptable excipients.

36. Formulations according to claim 35, wherein said one or more phenothiazinic compounds or derived compounds thereof present the Formula I: 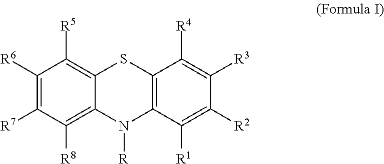
wherein R, R¹, R², R³, R⁴, R⁵, R⁶, R⁷ and R⁸ are independently selected from the group consisting of hydrogen, hydroxyl, halogen, alkyl, amine, amino, ketone, piperazine, trifluoromethyl, sulphanyl, piperidine, sulphynyl, azabicycle, pirrolidine, alkoxi, alkenyl, alkinyl, sulphidryl, amide, nitro, ciano and acyl wherein the cited substituents are and/or present substituted substituents or non-substituted, saturated or unsaturated and/or cyclic or of open chain.

37. Formulations according to claim 35 or 36, wherein said one or more phenothiazinic compounds or derived compounds thereof are selected from the group consisting of phenothiazine, chlorpromazine, promazine, trimeparzine, propiomazine, triflupromazine, etopropazine, prometazine, trifluoperazine (TFP), fluphenazine (FP), prochlorperazine, perphenazine, thiethylperazine, acetophenazine, carphenazine, thioridazine (TR), mesoridazine, mequitazine and metdilazine.

38. Cosmeceutical formulations according to claim 37, wherein the said one or more phenothiazinic compounds or derived compounds thereof are in the form of stabilized cation radicals.

39. Cosmeceutical formulations according to claim 38, wherein the said one or more phenothiazinic compounds or derived compounds thereof are in the monomeric or polymeric form.

40. Cosmeceutical formulations according to claim 38, wherein the said one or more phenothiazinic compounds or derived compounds thereof are in the form of pre-micellic aggregates and/or micelles.

41. Processes according to claim 38, wherein said one or more phenothiazinic compounds or derived compounds thereof are present in concentration raging from around 5 μM to around 2.5 mM.

42. Formulations according to claim 38, wherein formulation pH varies from 4.0 to 8.0.

43. Cosmeceutical formulations according to claim 38, wherein the said cosmeceutically acceptable excipients are selected from the group consisting of vehicle, agglutinants, desintegrants, binders, lubricants, surfactants, solubilizers, suspending agents, thickeners, diluents, solvents, emulsifiers, stabilizers, preservatives, colorants, seasonings, combinations thereof and others.

44. Cosmeceutical formulations according to claim 38, wherein also comprise cosmeceutical assistants selected from the group consisting of solar protection agents, fragrance agents, antibacterial agents, insect repellent agents, vitaminic agents, antioxidant agents, emollient agents, pH correction agents, combination thereof and others.

45. Cosmeceutical formulations according claim 38, wherein the said one or more phenothiazinic compounds or derived compounds thereof are comprised in dispersions, emulsions, pastes, powders, solutions, creams, colloids, gels, oils, macrocapsules, microcapsules, nanocapsules, macrospheres, microspheres, nanospheres, liposomes, oleosomes, chylomicrons, macroparticles, microparticles, nanoparticles, macrosponges, microsponges, nanosponges and others, or are found adsorbed in organic polymeric powders, talcs, bentonites and other organic or inorganic supports.

46. Cosmeceutical formulations according to claim 38, wherein the said cosmeceutical formulations are comprised in macrocapsules, microcapsules, nanocapsules, macrospheres, microspheres, nanospheres, liposomes, oleosomes, chylomicrons, macroparticles, microparticles, nanoparticles, macrosponges, microsponges, nanosponges and others, or are found adsorbed in organic polymeric powders, talcs, bentonites and other organic or inorganic supports, or are found in the form of dispersions, emulsions, pastes, powders, solutions, creams, colloids, serum, gels, oils, cream-gel, oil-gel, lotions, bases, ointments, unguents, milks, suspensions, foam, sprays, roll-on, sticks, lipsticks, patches and others.

47. Cosmeceutical formulations according to claim 38, wherein said cosmeceutical formulations are used in the prevention of skin diseases and disturbances.

48. Cosmeceutical formulations according to claim 38, wherein the said skin diseases and disturbances are selected from the group consisting of aging, wrinkles, skin rashes, drying, oxidation, burns, erythemas, dermatoses, dermatites, cancers and others.

49. Method of treating or preventing skin diseases or disturbances in a person having need of such treatment by applying to the skin a composition containing,
one or more phenothiazinic compounds or derived compounds thereof, wherein said compounds present the main structure: 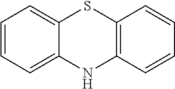
and a pharmaceutically acceptable vehicle.

50. The method of claim 49, wherein said one or more phenothiazinic compounds or derived compounds thereof present the Formula I: 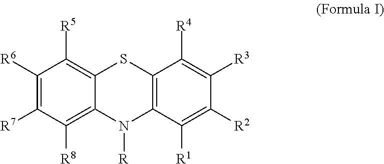
wherein R, R¹, R², R³, R⁴, R⁵, R⁶, R⁷ and R⁸ are independently selected from the group consisting of hydrogen, hydroxyl, halogen, alkyl, amine, amino, ketone, piperazine, trifluoromethyl, sulphanyl, piperidine, sulphynyl, azabicycle, pirrolidine, alkoxi, alkenyl, alkinyl, sulphidryl, amide, nitro, ciano and acyl wherein the cited substituents are and/or present substituted substituents or non-substituted, saturated or unsaturated and/or cyclic or of open chain.

51. The method of claim 49, wherein said one or more phenothiazinic compounds or derived compounds thereof are selected from the group consisting of phenothiazine, chlorpromazine, promazine, trimeparzine, propiomazine, triflupromazine, etopropazine, prometazine, trifluoperazine (TFP), fluphenazine (FP), prochlorperazine, perphenazine, thiethylperazine, acetophenazine, carphenazine, thioridazine (TR), mesoridazine, mequitazine and metdilazine.

52. The method of claim 51, wherein said one or more phenothiazinic compounds or derived compounds thereof are selected from the group consisting of phenothiazine, chlorpromazine, promazine, trimeparzine, propiomazine, triflupromazine, etopropazine, prometazine, trifluoperazine (TFP), fluphenazine (FP), prochlorperazine, perphenazine, thiethylperazine, acetophenazine, carphenazine, thioridazine (TR), mesoridazine, mequitazine and metdilazine.

53. The method of claim 51, wherein said one or more phenothiazinic compounds or derived compounds thereof are in the form of stabilized cation radicals.
